(19) United States
(12) Patent Application Publication
(10) Pub. No.: US 2013/0189366 A1
(43) Pub. Date: Jul. 25, 2013

(54) DRUG DELIVERY SYSTEMS AND METHODS OF USE

(71) Applicants: The United States Government as Represented by the Department of Veterans Affairs, Washington, DC (US); Brown University, Providence, RI (US)

(72) Inventors: Roshni S. Rainbow, Quincy, MA (US); Edith Mathiowitz, Brookline, MA (US)

(73) Assignees: Brown University, Providence, RI (US); The United States Government as Represented by the Department of Veterans Affairs, Washington, DC (US)

(21) Appl. No.: 13/748,976

(22) Filed: Jan. 24, 2013

Related U.S. Application Data
(60) Provisional application No. 61/590,671, filed on Jan. 25, 2012.

(51) Int. Cl. A61K 9/50

(52) U.S. Cl. CPC A61K 9/5073
USPC 424/492; 424/400; 424/93.1; 424/490; 424/497; 424/493

(57) ABSTRACT

Disclosed herein are systems for delivery of one or more molecules (such as a drug, for example, a small molecule, polypeptide, and/or nucleic acid) to a subject, for example to a targeted location in the subject. In some embodiments, the delivery system includes an encapsulated inducing agent or repressing agent and a plurality of cells. In some examples, the inducing agent or repressing agent and the cells are each separately encapsulated. In other examples, the encapsulated inducing agent or repressing agent and the cells are co-encapsulated. The cells include one or more genes which are operably linked to an inducible promoter or a repressible promoter which is inducible or repressible by the encapsulated inducing agent or repressing agent, respectively. Methods of use of the delivery systems, for example to treat or inhibit a disease or disorder in a subject, are also disclosed.

### CROSS REFERENCE TO RELATED APPLICATION

This claims the benefit of U.S. Provisional Application No. 61/590,671, filed Jan. 25, 2012, which is incorporated herein by reference in its entirety.

### ACKNOWLEDGMENT OF GOVERNMENT SUPPORT

This invention was made with government support awarded by the Department of Veterans Affairs. The government has certain rights in the invention.

### FIELD

This disclosure relates to drug delivery systems, methods of preparing the systems, and uses thereof for treating disease in a subject.

### BACKGROUND

A variety of growth factors are necessary for wound healing, including PDGF, FGF, KGF, EGF, TGF-α, TGF-βs, IGFs, VEGF and GM CSF. These growth factors are needed differentially at different stages for wound healing. Conventional delivery methods include delivery as a bolus and polymeric-based delivery. However, conventional delivery methods compromise bioactivity of growth factors. Recombinant growth factors are usually available in an active form and thus are prone to denaturation and degradation. Entrapped active growth factors become intrinsically less potent over time, may lose bioactivity or may degrade during the fabrication process. As a result, larger amounts of growth factors are needed to achieve therapeutic efficacy. There is a need to develop systems for more effective delivery of bioactive compounds, such as growth factors, for various therapeutic or other uses.

### SUMMARY

Disclosed herein are systems for delivery of one or more molecules (such as a drug, for example, a small molecule, polypeptide, or nucleic acid) to a subject, for example to a targeted location in the subject. In some embodiments, the delivery system includes an encapsulated inducing agent or repressing agent and an encapsulated plurality of cells. In some examples, the inducing agent or repressing agent and the cells are each separately encapsulated. In other examples, the cells and the encapsulated inducing agent or repressing agent are co-encapsulated. The cells include one or more genes which are operably linked to an inducible promoter or a repressible promoter, which is inducible or repressible by the encapsulated inducing agent or repressing agent, respectively. In one non-limiting embodiment, the delivery system includes cells including an insulin-like growth factor 1 (IGF-1) gene operably linked to a tetracycline inducible promoter encapsulated in Ca²⁺-alginate microspheres or microcapsules and doxycycline encapsulated in poly(lactic-co-glycolic acid) (PLGA) nanospheres, microspheres or microcapsules. In some examples, the cells and PLGA nanospheres or microspheres are co-encapsulated with the cells in Ca²⁺-alginate microspheres or macrospheres (or capsules).

Also disclosed herein are methods of treating or inhibiting a disease or disorder in a subject by administering an effective amount of a disclosed delivery system to a subject. In one non-limiting example, a delivery system is administered in which encapsulated cells including an IGF-1 gene operably linked to a tetracycline inducible promoter and encapsulated tetracycline or a tetracycline analog to a subject.

Kits including the delivery system are also disclosed.

The foregoing and other features of the disclosure will become more apparent from the following detailed description, which proceeds with reference to the accompanying figures.

### BRIEF DESCRIPTION OF THE DRAWINGS

FIG. 1A-C is a series of schematics showing exemplary encapsulated delivery systems including co-encapsulation of the cells and inducing/repressing agent. FIG. 1A is a schematic illustrating exemplary co-encapsulation in a macrocapsule of drug loaded microspheres (such as DOX-loaded PLGA microspheres) and cells with drug-inducible gene expression (such as Tet-inducible growth factor expression). FIG. 1B is a schematic illustrating exemplary co-encapsulation in a macrocapsule of drug loaded microspheres and separately encapsulated cells with drug-inducible gene expression. FIG. 1C is a schematic illustrating co-encapsulation of a drug and encapsulated cells with inducible gene expression in a macrocapsule.

FIG. 2A is a graph showing cumulative IGF-1 release from unencapsulated normal human fibroblast (NHF) cells (open circles) and unencapsulated modified NHFs expressing human IGF-1 gene (closed circles). Cells were grown to confluence in E-well plates and medium was analyzed at the indicated time points. Mass released was normalized to 10⁶ viable cells at the completion of each study. Data were fit to first-order linear regression forced through the axis. All data are represented as mean±standard deviation (n=6).

FIG. 2B is a graph showing cumulative IGF-1 release from encapsulated unmodified NHFs (open circles) and encapsulated modified NHFs expressing human IGF-1 gene (closed circles). Methods and data analysis were as described for FIG. 2B. All data are represented as mean±standard deviation (n=12).

FIG. 2C is a digital image showing phase contrast microscopy of NHFs immobilized in Ca²⁺-alginate microcapsules. The image shows a cross-sectional view of one plane in the capsules. Capsules included 30-40 cells per capsule; volumetric diameter was about 500 μm. Arrows indicate cells in a contracted morphology. Scale bar=200 μm.

FIG. 3 is a graph showing doxycycline (DOX)-induced cumulative release of IGF-1 from unencapsulated CHO-K1 Tet-On cells (open circles) and unencapsulated CHO-K1 Tet-On cells expressing IGF-1 (CHO-K1 Tet-IGF1 cells) in response to 12 hour exposure to 1 μg/ml DOX. Release was normalized to 10⁶ viable cells at the end of the study for each cell type. All data are represented as mean±standard deviation (n=6).

FIG. 4 is a graph showing IGF-1 release as a function of DOX concentration by encapsulated (closed circles) or unencapsulated (open circles) CHO-K1 Tet-IGF1 cells over 24 hours. IGF-1 release was measured and normalized to 10⁶ viable cells counted at the end of the study for each cell type. All data are represented as mean±standard deviation (n=3).

FIG. 5 is a graph showing DOX-modulated release of IGF-1 from encapsulated CHO-K1 Tet-IGF1 cells (closed circles) or encapsulated CHO-K1 Tet-On cells (open circles) cultured at 32° C. Cells were continuously exposed to 1 μg/mL DOX for 3 days, after which DOX was completely removed. IGF-1 release was measured daily and normalized to 10⁶ viable cells counted at the end of the study. All data are represented as mean±standard deviation (n=11).

FIG. 6A is a graph showing cumulative fractional release of DOX from loaded (closed circles) or unloaded (open circles) PLGA microspheres for the first 24 hours of an 85 day DOX exposure study. Release was measured using UV spectrophotometry and expressed as the fractional cumulative release normalized to the total cumulative mass released on the last day of the study (n=6).

FIG. 6B is a graph showing cumulative fractional release of DOX from loaded (closed circles) or unloaded (open circles) PLGA microspheres over an 85 day DOX exposure study. Release was measured using UV spectrophotometry and expressed as the fractional cumulative release normalized to the total cumulative mass released on the last day of the study (n=6).

FIG. 7A-B is a pair of digital images of phase contrast microscopy of a cross-section of Ca2+-alginate macrocapsules containing only PLGA microspheres (FIG. 7A) or containing both separate CHO-K1 Tet-IGF1 cells and PLGA microspheres (FIG. 7B). The inset shows the boxed area at higher power with the arrow indicating cells in a contracted state (inset scale bar=50 μm).

FIG. 8 is a graph showing IGF-1 release from unencapsulated or encapsulated CHO-K1 Tet-IGF1 cells stimulated by DOX released from PLGA microspheres or delivered as a bolus dosage (n=3). In both formats, the total amount of DOX delivered to the cells was 1 μg/mL at the end of the 24-hour culture. Cultures exposed to unloaded PLGA microspheres (blank spheres) or no DOX served as the positive and negative controls, respectively.

FIG. 9A is a graph showing DOX release profile from co-encapsulated CHO-K1 Tet-IGF1 cells and DOX-loaded PLGA microspheres shown as concentration (top) or normalized to the number of capsules per sample (bottom).

FIG. 9B is a graph showing IGF-1 release profile from co-encapsulated CHO-K1 Tet-IGF1 cells and DOX-loaded PLGA microspheres shown as concentration (top) or normalized to the number of capsules per sample (bottom). Released IGF-1 was normalized to 10⁶ encapsulated cells per sample at the end of the study (top) or to the number of capsules per sample (bottom).

### DETAILED DESCRIPTION

Disclosed herein are systems for targeted delivery of one or more therapeutic compounds (such as a drug, for example a small molecule, polypeptide, or nucleic acid) to a subject. In some embodiments, the disclosed systems allow local synthesis and/or release of the therapeutic compound(s), allowing targeted delivery of the compound in both time and space. For example, in some embodiments, the disclosed delivery systems include encapsulated cells which include a gene operably linked to a regulatable promoter (such as an inducible or repressible promoter) and an encapsulated inducing agent or repressing agent which can regulate (for example, induce or repress) expression of the gene from the inducible or repressible promoter. In some embodiments, the encapsulated cells and the encapsulated inducing or repressing agent are separately encapsulated in distinct capsules and can be administered to the same or substantially the same location in the subject and release and diffusion of the inducing or repressing agent results in regulation of gene expression by the encapsulated cells. In other embodiments, the cells (for example, unencapsulated cells or encapsulated cells) and encapsulated inducing or repressing agent are co-encapsulated in a single capsule matrix. One particular advantage of the disclosed systems is that they permit localized control of gene expression without systemic administration of an inducing or repressing agent to the subject. An additional advantage is that the system surprisingly provides both sustained release and increased amounts of the inducible gene product as compared to bolus administration of an equivalent amount of the inducing agent.

### I. Abbreviations

DCM dichloromethane

DOX doxycycline

EGF epidermal growth factor

FGF fibroblast growth factor

GF growth factor

GM-CSF granulocyte macrophage colony stimulating factor

IGF insulin-like growth factor

KGF keratinocyte growth factor

NHF normal human fibroblast

PDGF platelet derived growth factor

PLGA poly(lactic-co-glycolic acid)

SEM scanning electron microscopy

Tet tetracycline

TGF transforming growth factor

VEGF vascular endothelial growth factor

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. As used herein, “comprises” means “includes.” Thus, “comprising A or B,” means “including A, B, or A and B,” without excluding additional elements. It is further to be understood that all base sizes or amino acid sizes, and all molecular weight or molecular mass values, given for nucleic acids or polypeptides are approximate, and are provided for description. All publications, patent applications, patents, and other references mentioned herein are incorporated by reference in their entirety for all purposes.

Although methods and materials similar or equivalent to those described herein can be used in the practice or testing of the present disclosure, suitable methods and materials are described below. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Drug: A compound or composition that in some examples, is effective to treat or inhibit a disorder in a subject (such as a therapeutically effective compound or composition). A drug can include any type of compound or composition, including but not limited to small molecules, polypeptides, and nucleic acids.

Encapsulate: As used herein, a compound or cell “encapsulated” in a particle (such as a nanoparticle or microparticle; also referred to as a nanosphere, nanocapsule, microsphere, or microcapsule) refers to a compound or cell that is either contained within the particle or attached to the surface of the particle, or a combination thereof. Co-encapsulated includes one or more different compounds and/or cells that are contained within the same particle (such as the same microparticle, microsphere, microcapsule, or the same macroparticle, macrosphere, or macrocapsule).

Effective amount: An amount of a compound or a combination of compounds sufficient to cause the desired effective, for example an amount sufficient to treat or inhibit a disease or condition in a subject. The amount of a compound or combination of compounds which is an effective amount will vary depending on the compound, the disease or condition and its severity, the age of the subject, and so on. An effective amount can be determined by one of ordinary skill in the art.

Growth factor: A molecule (such as a polypeptide) that modulates (for example, increases or decreases) one or more of cell growth, division, proliferation, survival, or differentiation. Growth factors include, but are not limited to epidermal growth factor (EGF), fibroblast growth factors (FGF, including FGF1 to 10), granulocyte macrophage colony stimulating factor (GM-CSF), insulin-like growth factors (IGF, including IGF-1 and IGF-2), keratinocyte growth factor (KGF), platelet-derived growth factor (PDGF), transforming growth factors (TGF, including TGF-α and TGF-βs), and vascular endothelial growth factors (VEGF). One of ordinary skill in the art can identify additional growth factors.

Expression: The process by which the coded information of a gene is converted into an operational, non-operational, or structural part of a cell, such as the synthesis of a nucleic acid or protein. Gene expression can be influenced by external signals. For instance, exposure of a cell to a hormone may stimulate expression of a hormone-induced gene. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

Inducible/Inducing: To increase (for example, significantly increase expression) of a gene, for example, by activating or increasing transcription of a gene. A gene or promoter is inducible if its expression or activity can be activated or increased by binding of a transcription activator. In one example, an inducible promoter is a Tet-inducible promoter, wherein expression of a gene linked to the promoter can be activated or increased by binding of tetracycline or a Tet analog to a tetracycline responsive transcription activator. An inducing agent includes a compound (such as a polypeptide or small molecule) that increases or activates transcription of a gene operably linked to an inducible promoter which is activated by a transcription activator responsive to the inducing agent.

Inhibiting or treating a disease: “Inhibiting” a disease or disorder refers to inhibiting the full development of a disease, for example in a person who is known to have a disease or be at risk for developing a disease. Inhibition of a disease or disorder can span the spectrum from partial inhibition to substantially complete inhibition (or even prevention) of the disease or disorder. In some examples, the term “inhibiting” refers to reducing or delaying the onset or progression of a disease or disorder. In other examples, inhibiting a disease refers to lessening symptoms of the disease or disorder. A subject to be administered an effective amount of a composition to inhibit or treat the disease or disorder can be identified by standard diagnosing techniques for such a disorder, including, for example, basis of family history, or risk factors to develop the disease or disorder. “Treatment” refers to a therapeutic intervention that ameliorates a sign or symptom of a disease or pathological condition after it has begun to develop.

Operably linked: A first nucleic acid is operably linked with a second nucleic acid when the first nucleic acid is placed in a functional relationship with the second nucleic acid. For instance, a promoter is operably linked to a coding sequence if the promoter affects the transcription or expression of the coding sequence. Generally, operably linked nucleic acids are contiguous and, where necessary to join two protein-coding regions, in the same reading frame. In some examples, a promoter is operably linked to a protein encoding nucleic acid, such that the promoter drives transcription of the linked nucleic acid and/or expression of the protein.

Particle: A carrier having a matrix, such as a polymer matrix, in which smaller particles or compounds can be contained. The particles can be a variety of shapes, but are typically spherical.

Pharmaceutically acceptable carriers: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the agents disclosed herein.

In general, the nature of the pharmaceutically acceptable carrier will depend on the particular mode of administration being employed. For instance, parenteral or implantable formulations usually comprise fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: Promoters are sequences of DNA near the 5′ end of a gene that act as a binding site for RNA polymerase, and from which transcription is initiated. A promoter includes necessary nucleic acid sequences near the start site of transcription, such as, in the case of a polymerase II type promoter, a TATA element. In one embodiment, a promoter includes an enhancer. In another embodiment, a promoter includes a repressor element.

Promoters may be constitutively active, such as a promoter that is continuously active and is not subject to regulation by external signals or molecules. In some examples, a constitutive promoter is active such that expression of a nucleic acid operably linked to the promoter is expressed ubiquitously (for example, in all cells of a tissue or in all cells of an organism and/or at all times in a single cell or organism, without regard to temporal or developmental stage).

Promoters may be inducible or repressible, such that expression of a nucleic acid operably linked to the promoter can be expressed under selected conditions. In some examples, a promoter is an inducible promoter, such that expression of a nucleic acid operably linked to the promoter is activated or increased. An inducible promoter may be activated by presence or absence of a particular molecule, for example, doxycycline, tetracycline, metal ions, alcohol, or steroid compounds. An inducible promoter also includes a promoter that is activated by environmental conditions, for example, light or temperature. In further examples, the promoter is a repressible promoter such that expression of a nucleic acid operably linked to the promoter can be reduced to low or undetectable levels, or eliminated. A repressible promoter may be repressed by direct binding of a repressor molecule (such as binding of the trp repressor to the trp operator in the presence of tryptophan). In a particular example, a repressible promoter is a tetracycline repressible promoter. In other examples, a repressible promoter is a promoter that is repressible by environmental conditions, such as hypoxia or exposure to metal ions.

Repress/repressible: To inhibit or significantly reduce expression, for example, by inhibiting (or even preventing) or blocking transcription of a gene. A gene or promoter is repressible if its expression or activity can be reduced or suppressed by inhibiting binding of a transcription activator. In one example, a repressible promoter is the Tet-repressible promoter, wherein expression of a gene linked to the promoter can be inhibited or reduced by binding of tetracycline or a Tet analog to a tetracycline responsive transcription activator. A repressing agent includes a compound (such as a peptide or small molecule) that decreases or inhibits transcription of a gene operably linked to a repressible promoter which is repressed by binding of the repressing agent to a transcription activator responsive to the repressing agent.

Subject: Living multi-cellular vertebrate organisms, a category that includes both human and non-human mammals. In some examples, a subject is a mammal, such as a human, non-human primate, rodent, horse, cow, or other agricultural or domesticated animal.

### III. Delivery System

Disclosed herein are systems for delivery of one or more molecules (such as a drug, for example, a small molecule, polypeptide, and/or nucleic acid) to a subject, for example to a targeted location in the subject. In some embodiments, the delivery system includes an encapsulated inducing agent or repressing agent and an encapsulated plurality of cells. In some examples, the inducing agent or repressing agent and the cells are each separately encapsulated. In other examples, the cells and the encapsulated inducing agent or repressing agent are co-encapsulated. In other embodiments agents (for example an unencapsulated agent or an encapsulated agent, such as an agent encapsulated in a microsphere or microcapsule) or cells (such as unencapsulated or encapsulated cells) are further encapsulated in a macrocapsule. In one embodiment, microspheres of an encapsulated agent (such as PLGA encapsulated DOX) are co-encapsulated in a macrocapsule with cells (such as cells including a Tet-inducible gene). The cells include one or more genes which are operably linked to promoter inducible by the inducing agent (for example, an inducible promoter) or a promoter repressible by the repressing agent (for example, a repressible promoter), such that the encapsulated inducing agent or repressing agent modifies (for example induces or represses, respectively) expression of the gene operably linked to the promoter.

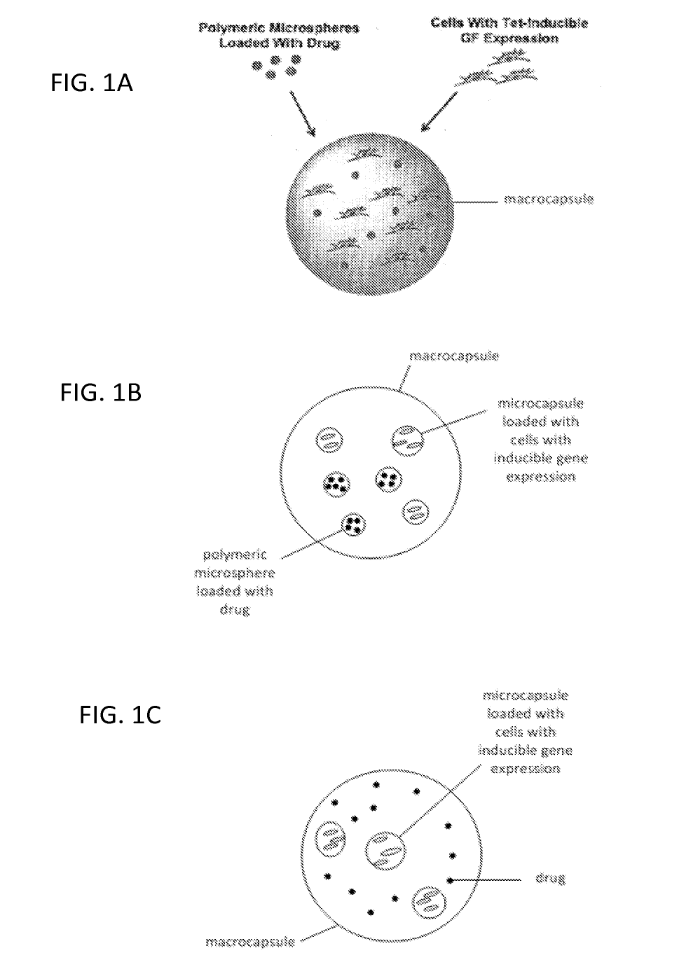

The encapsulated cells and encapsulated inducing (or repressing) agent can exist together in the same local area, with the inducing (or repressing) agent diffusing to the encapsulated cells, or the cells and encapsulated inducing (or repressing) agent can exist in a single implantable system (for example, are co-encapsulated, e.g., FIG. 1A). In both examples, the need for systemic or bolus delivery of the inducing (or repressing) agent is reduced or even eliminated due to the targeted co-localization of the elements of the system. Release of the drug from the microspheres over time induces gene expression by the cells and the induced gene product (such as a growth factor) is gradually released from the macrocapsule by diffusion. In some examples, the materials used for encapsulation of the cells, inducing (or repressing) agent, and the co-encapsulation (if applicable) are the same material (for example the same polymer). In other examples, the encapsulation material is different for at least one (or each) component. In one example, the inducing or repressing agent is encapsulated with a polymer or copolymer such as PLGA and the cells and/or the co-encapsulation material is a hydrogel, such as alginate. Additional suitable encapsulation materials are discussed below.

FIG. 1A illustrates an exemplary co-encapsulated delivery system which is a macrocapsule made of a matrix (such as a polymer or hydrogel) that contains cells having for example a tet-inducible gene (such as a growth factor gene) and also contains polymeric microspheres (such as PLGA microspheres) containing a drug such as an inducing or repressing agent. Other exemplary encapsulation systems are illustrated in FIGS. 1B and C. FIG. 1B illustrates another exemplary co-encapsulated delivery system which is a macrocapsule made of a matrix (such as a polymer or hydrogel) that contains cells having for example a tet-inducible gene (such as a growth factor gene), which are themselves encapsulated in a microcapsule, such as an alginate microcapsule. The macrocapsule also contains separately encapsulated drug (such as an inducing or repressing agent) which is contained in polymeric microspheres. FIG. 1C is an additional exemplary co-encapsulated delivery system which is a macrocapsule made of a matrix (such as a polymer or hydrogel) that contains cells having for example a tet-inducible gene (such as a growth factor gene), which are themselves encapsulated in a microcapsule. The macrocapsule also contains a drug (such as an inducing or repressing agent).

Some embodiments of the disclosure include a dual delivery system having a plurality of polymeric-based microspheres encapsulating an inducing agent (or repressing agent), and a plurality of cells genetically engineered to express an inducible gene upon induction by the inducing agent (or to decrease or inhibit expression of a repressible gene upon repression by the repressing agent) such that the cells are encapsulated within a first polymeric-based microcapsule, such that each of the microspheres is co-encapsulated with each of the microcapsules within a second polymeric-based microcapsule and the gene is expressed (or repressed) upon release of the inducing agent (or repressing agent) from the microspheres. For example, the microspheres can include PLGA-based microspheres.

The disclosed delivery systems include a plurality of cells including a gene of interest operably linked to a regulatable promoter. In some examples, the plurality of cells includes a population of cells including the gene of interest. In other examples, the plurality of cells includes two or more populations of cells which each include a different gene of interest operably linked to a regulatable promoter (such as 2, 3, 4, 5, or more populations of cells). The genes of interest may be linked to the same regulatable promoter (for example, a tetracycline inducible or repressible promoter) or may be linked to different regulatable promoters. In some examples, the two or more populations of cells are co-encapsulated. In other examples, the two or more populations of cells are each separately encapsulated.

The disclosed delivery systems also include an encapsulated inducing or repressing agent. In some examples, the encapsulated inducing (or repressing) agent includes one or more inducing (or repressing) agents (for example, 1, 2, 3, 4, 5, or more). In some examples, the two or more inducing or repressing agents are co-encapsulated. In other examples, the two or more inducing or repressing agents are each separately encapsulated.

A. Encapsulation

The disclosed system includes encapsulated inducing or repressing agents and/or encapsulated cells. Various types of biodegradable and biocompatible polymers can be used for encapsulation, and methods of encapsulating a variety of synthetic compounds, cells, proteins, and nucleic acids have been well described in the art. See, for example, U.S. Pat. Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006; all of which are incorporated by reference herein.

In some embodiments, agents or cells are encapsulated in nanospheres (nanocapsules), microspheres (microcapsules), or macrospheres (macrocapsules) which are composed of a polymer. In some examples, the polymer includes copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. In one example, the polymer is 50:50 PLGA copolymer. In other examples, the polymer includes a natural polymer, such as chitosan, collagen, alginate, gelatin, hyaluronic acid, and nontoxic metal salts thereof. In some embodiments the polymer is a hydrogel, for example an alginate hydrogel. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The agents or cells can be combined with the polymer according to a variety of methods, and release of the agents or compounds synthesized by the cells can be by diffusion, disintegration of the vehicle, or associated formation of water channels. In some circumstances, the agent is dispersed in macrocapsules (macrospheres), microcapsules (microspheres) or nanocapsules (nanospheres) prepared from a suitable polymer, for example, PLGA, and dispersed in a biocompatible dispersing medium, which yields sustained delivery and biological activity over a protracted time.

In some embodiments, encapsulated substances (for example, small molecules (such as DOX) and/or cells) are produced using solvent removal methods, such as single or double emulsion methods, phase separation, or spray drying. In some examples, encapsulation is carried out with a single emulsion method, for example where a polymer is dissolved in an organic solvent (including, but not limited to dichloromethane) and a substance (such as a drug) is dissolved or suspended in the polymer solution. The mixture is emulsified in water (W/O) to form spheres and then the solvent is removed (for example, by evaporation or extraction in water). In other examples, such as when the substance to be encapsulated is hydrophobic, a hydrophobic oil is used in the emulsion (O/O) instead of water.

In additional examples, a double emulsion method is utilized, for example where an aqueous solution of a water-soluble compound is emulsified with polymer dissolved in an organic solvent to form a W/O emulsion using homogenizer or sonicator. This primary emulsion is transferred to water (W/O/W) or an oil (W/O/O) and stirred or sonicated to form a secondary emulsion. The solvent is removed by evaporation, extraction, or precipitation to obtain the spheres. In some examples, the aqueous solution containing the substance being encapsulated may contain salt (for example NaCl) at about 0.1%-10% (for example, about 0.5%-5%, about 0.1%-1%, about 1%-10%, about 1%-2%, such as about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10%). Exemplary encapsulation methods are described in the Examples below.

In other examples, encapsulation methods include phase separation, which involves dissolving a polymer in an organic solvent and dispersing or dissolving a substance in the polymer solution. An organic non-solvent is added with continuous mixing and the polymer solvent is gradually extracted. In another type of phase separation method, polymer solution droplets and drug or other compound solution droplets are allowed to collide (for example using ink-jet nozzles). The polymer containing droplet spreads over the aqueous droplet, producing a reservoir type droplet. Spray drying may also be used to form spheres. A compound is dissolved or dispersed in a polymer solution, which is sprayed in a stream of heated air to produce microparticles.

In some examples, macrocapsules, such as those disclosed herein, have a length of about 1-10 cm and an inner diameter of about 0.5-1.5 mm. Macrocapsules can be spherical, but any convenient shape (such as flat sheet diffusion chambers) can be used. In some examples, macrocapsules can include between 10 and 10⁸ cells per macrocapsule. Microcapsules (or microspheres), such as those disclosed herein, are smaller than macrocapsules, and in some examples are spheres with an inner diameter of about 200-800 μm. Typically, microcapsules can hold about 10 to 10³ cells per capsule. In further examples, nanospheres (or nanocapsules) have an inner diameter of less than 200 μm, for example, about 10-150 μm.

Encapsulated cell devices typically consist of three components: an immunoisolatory membrane, an internal matrix, and the cells themselves. A wide variety of biocompatible materials are useful for fabricating immunoisolatory membranes, with synthetic thermoplastics and polymer blends typically being utilized in all extracorporeal and some macrocapsule-based devices. In other examples, microcapsules and a small fraction of macrocapsule designs utilize hydrogel-based polymers. All designs typically incorporate some internal matrix within the membranes, analogous to native extracellular matrix, to provide the encapsulated cells with physical scaffolding that supports cell viability and regulates cellular function. In some embodiments the matrix, which may be a hydrogel or solid scaffold, insures even dispersion of the cells within the device, thereby preventing cell aggregation and any associated diffusion limitations and central necrosis. In some examples, devices utilizing synthetic polymer membranes are preformed, loaded with an internal matrix and cells, and then sealed. In other examples, designs utilizing hydrogel-based membranes usually require the cells to be present during the fabrication process. In one particular example, calcium alginate hydrogel is utilized as both the membrane and matrix material for cell encapsulation in both micro- and macrocapsule formats. Alginate, a natural polymer widely used in the field of encapsulated cell technologies, is a marine-derived polysaccharide composed of chains of β-D-mannuronic acid and α-L-guluronic acid residues. These chains are capable of cross-linking through interactions with divalent metal ions, such as Ca²⁺ or Ba²⁺, to create a hydrogel structure. If cells are present in the alginate prior to crosslinking, they become immobilized within the forming hydrogel during the crosslinking reaction. The release of therapeutics synthesized by these encapsulated cells can be adjusted by varying the encapsulated cell density and cross-linking properties of alginate. An advantage of alginate microcapsules or macrocapsules is that they are biocompatible and can maintain stability for long periods of time, in part due to the highly hydrated three-dimensional extracellular matrix-like structure.

B. Genes and Regulatable Expression Systems

The disclosed delivery systems include cells which include a gene that is operably linked to a regulatable promoter (such as an inducible promoter or a repressible promoter). In some embodiments, the gene encodes a therapeutically effective compound, the expression of which is regulated (for example increased or decreased) in the presence of a suitable inducing or repressing agent. One of ordinary skill in the art can select a gene for inclusion in the cells of the disclosed delivery systems based on the desired effect, for example the particular disease or disorder to be treated.

In some embodiments, the gene encodes a growth factor. In some examples, the growth factor is a molecule (such as a polypeptide) that modulates (for example, increases or decreases) one or more of cell growth, division, proliferation, survival, or differentiation. Growth factors include molecules involved in regulation of wound healing, angiogenesis, tumor growth and/or metastasis, hematopoiesis, and many other cellular and physiological processes. In some examples, growth factors include, but are not limited to epidermal growth factor (EGF), fibroblast growth factors (FGF, including FGF1 (acidic FGF), FGF2 (basic FGF), FGF3, FGF4, FGF5, FGF6, FGF7 (keratinocyte growth factor; KGF), FGF8, FGF9, and FGF10), granulocyte macrophage colony stimulating factor (GM-CSF), insulin-like growth factors (IGF, including IGF-1 and IGF-2), platelet-derived growth factors (PDGF, including PDGFA, PDGFB, PDGFC, and PDGFD), transforming growth factors (TGF, including TGF-α and TGF-βs (for example, TGF-β1, TGF-β2, and TGF-β3)), and vascular endothelial growth factors (VEGF; including VEGF-A, VEGF-B, VDGF-C, VEGF-D, and placenta growth factor). Additional growth factors include bone morphogenetic proteins (BMP; including BMPs 1-20), brain-derived neurotrophic factor (BDNF), glial cell line-derived neurotrophic factor (GDNF), ciliary neurotrophic factor (CNTF), nerve growth factor (NGF), hepatocyte growth factor (HGF), and tumor necrosis factor-α (TNF-α). One of ordinary skill in the art can identify additional growth factors.

In other examples, the gene encodes a therapeutic or potentially therapeutic peptide that is not typically considered a growth factor. In some examples, the gene encodes insulin. In other examples, the gene encodes a cytochrome P450, a cytokine (such as an interleukin or interferon, for example interleukin-2), or growth hormone. Any gene encoding a protein or a portion thereof can be included in the disclosed systems. In other examples, the gene includes an antisense oligonucleotide (such as an siRNA or shRNA). One of ordinary skill in the art can select a gene for inclusion in the system based on the desired use.

In some embodiments, the encapsulated cells disclosed herein include a gene operably linked to an inducible promoter and expression of the gene is activated upon exposure to an appropriate inducing agent. Inducible promoters are known to one of ordinary skill in the art. In some examples, the inducible promoter is a mammalian inducible promoter. In one example, an inducible promoter is a Tet-inducible promoter, wherein expression of a gene linked to the promoter can be increased by binding of tetracycline or a Tet analog (such as DOX) to a tetracycline responsive transcription activator. In some examples, a cell line is doubly transfected or transformed with a vector including the Tet-responsive transcription activator and a vector including the gene of interest operably linked to a Tet inducible promoter. Expression of the gene of interest is induced in the presence of Tet or a Tet analog. In other examples, a cell line is doubly transfected or transformed with a vector encoding a tetracycline repressor and a vector including a gene of interest operably linked to a promoter including a Tet operator site. Expression of the gene of interest is repressed in the absence of Tet and is de-repressed (induced) in the presence of Tet or a Tet analog. Suitable vectors are commercially available and include the Tet-On™ tetracycline inducible expression system or Tet-Express™ inducible expression system (Clontech, Mountain View, Calif.) and T-Rex™ inducible expression system (Invitrogen, Carlsbad, Calif.). Inducing agents for use with tetracycline inducible promoters and expression systems include tetracycline and tetracycline analogs (such as doxycycline or anhydrotetracycline).

Additional inducible expression systems include an ecdysone inducible system or a “Rheo” receptor inducible system. Suitable systems are commercially available and include the ecdysone inducible Complete Control inducible mammalian expression system (Agilent Technologies, Santa Clara, Calif.) and the RHEOSWITCH® mammalian inducible expression system (New England BioLabs, Ipswich, Mass.). Inducing agents for use with ecdysone inducible promoters and expression systems include ecdysone and ecdysone analogs (such as ponasterone A). Inducing agents for use with a Rheo receptor inducible promoter and expression system includes a Rheo receptor ligand (such as RSL1, New England BioLabs). Additional inducible gene expression systems include macrolide-based inducible systems (e.g., Weber et al., Nature Biotechnol. 20:901-907, 2002) and streptogramin-based inducible gene expression systems (e.g., Fussenegger et al., Nature Biotechnol. 18:1203-1208, 2000). Inducing agents for use with macrolide-based inducible systems include erythromycin, clarithromycin, and roxithromycin. Inducing agents for use with streptogramin-based inducible systems include pristinamycin, virginiamycin, and quinupristin/dalfopristin.

In other embodiments, the encapsulated cells disclosed herein include a gene operably linked to a repressible promoter and expression of the gene is repressed (for example, reduced or inhibited) upon exposure to an appropriate repressing agent. Repressible promoters are known to one of ordinary skill in the art. In some examples, the repressible promoter is a mammalian repressible promoter. In one example, a repressible promoter is a Tet-repressible promoter, wherein expression of a gene linked to the promoter is repressed or inhibited by binding of tetracycline or a Tet analog (such as DOX) to a tetracycline responsive transcription activator which is active (promotes gene expression) in the absence of Tet, but which is not active in the presence of Tet or a Tet analog. Tet repressible expression systems are commercially available and include the Tet-Off™ inducible gene expression systems (Clontech, Mountain View, Calif.). Repressing agents for use with tetracycline repressible promoters and expression systems include tetracycline and tetracycline analogs (such as doxycycline or anhydrotetracycline).

Additional repressible gene expression systems include macrolide-based repressible systems (e.g., Weber et al., Nature Biotechnol. 20:901-907, 2002) and streptogramin-based repressible gene expression systems (e.g., Fussenegger et al., Nature Biotechnol. 18:1203-1208, 2000). Repressing agents for use with macrolide-based repressible systems include erythromycin, clarithromycin, and roxithromycin. Repressing agents for use with streptogramin-based repressible systems include pristinamycin, virginiamycin, and quinupristin/dalfopristin).

C. Cells

Cells of use in the disclosed systems and methods include any cells that can be modified to introduce the desired gene operably linked to an inducible promoter or a repressible promoter. Methods for introducing exogenous nucleic acids into cells (for example by transformation or transfection) are known to one of ordinary skill in the art and are routine. The cells include immortalized or primary cell lines, particularly mammalian cell lines. In some examples, the cells are human cells; however other mammalian cells, including mouse, hamster, or canine cells can also be used. Some particular, non-limiting examples include Chinese hamster ovary (CHO) cell line, retinal pigment endothelial cell lines (e.g., ARPE-19), baby hamster kidney (BHK) cell line, human embryonic kidney cell lines (e.g., HEK293), fibroblast cells or cell lines (such as primary fibroblasts (for example normal human fibroblasts) or a fibroblast cell line (for example, murine fibroblast 3T3 cells)), myoblast cells or cell lines (such as murine C2C12 mouse myoblast cell line), primary hepatocytes, primary renal proximal tubule cells, primary parathyroid cells, benign insulinoma cells, pancreatic islet cells, or adrenal chromaffin tissue. One of ordinary skill in the art can select additional cells or cell lines suitable for the disclosed systems and methods, for example, based on the gene to be included, the subject, and/or the disease or disorder to be treated. Cells and cell lines are commercially available (for example, from the American Type Culture Collection, Manassas, Va.).

### IV. Methods of Treatment

The delivery systems disclosed herein can be used to deliver one or more therapeutic agents (such as one or more drugs, for example, one or more small molecules, nucleic acids, polypeptides, or a combination of two or more thereof) to a subject, for example to treat or inhibit a disease or disorder in the subject. In some examples, an effective amount of the delivery system is administered to the subject, for example in an amount sufficient to decrease one or more sign or symptom of the disease or disorder or to inhibit onset or progression of the disease or disorder.

In some examples, the disclosed methods include administering an effective amount of a delivery system (such as encapsulated inducing or repressing agent, encapsulated cells, and/or co-encapsulated inducing (or repressing) agents and cells) to a subject with a degenerative joint disease (such as osteoarthritis or end-stage rheumatoid arthritis) or a bone injury (such as a bone fracture). In some examples, a delivery system including an encapsulated inducing agent and one or more encapsulated cells including a growth factor operably linked to an inducible promoter (co-administered or co-encapsulated) is administered to the subject. In particular non-limiting examples, the delivery system includes at least an encapsulated plurality of cells including an IGF-1 gene operably linked to a tetracycline inducible promoter and encapsulated doxycycline. In one example, the cells and encapsulated DOX are co-encapsulated. In some examples, the delivery system can additionally include encapsulated cells including a TGF-β2 or FGF-2 gene operably linked to a tetracycline inducible promoter.

In other examples, the methods include administering an effective amount of the delivery system to a subject with a neurodegenerative disease (for example, Parkinson's disease, Alzheimer's disease, amyotrophic lateral sclerosis, Huntington's disease, or spinal muscular ataxia), retinal degeneration (such as retinitis pigmentosa) or macular degeneration, diabetes, a tumor or metastasis, organ damage or failure (such as liver or kidney damage or failure), hypoparathyroidism, or chronic pain. One of ordinary skill in the art can select the appropriate gene(s) for the condition being treated. For example, the delivery system would include encapsulated cells including an insulin gene operably linked to a regulatable promoter for the treatment of diabetes.

In some examples, the amount of encapsulated inducing (or repressing) agent and encapsulated cells administered to the subject includes a specified number of cells (for example, about 1×10⁶ cells or more, such as about 1×10⁶ to about 1×10¹¹ cells, such as about 1×10⁶ cells, 5×10⁶ cells, 1×10⁷ cells, 5×10⁷ cells, 1×10⁸ cells, 5×10⁸ cells, 1×10⁹ cells, 5×10⁹ cells, 1×10¹⁰ cells, 5×10¹⁰ cells, or 1×10⁶ to 1×10⁹ cells, 1×10⁶ to 1×10¹⁰ cells, 1×10⁷ to about 1×10¹⁰ cells, 1×10⁷ to 1×10⁹ cells). In other examples, the amount of encapsulated inducing (or repressing) agent and encapsulated cells administered to the subject includes a pre-determined percentage of loading of the capsules with the inducing or repressing agent (such as about 0.1% to about 20% w/w loading or more, for example, about 0.2%, 0.3%, 0.4%, 0.5%, 0.6%, 0.7%, 0.8%, 0.9%, 1%, 1.5%, 2%, 2.5%, 3%, 4%, 5%, 6%, 7%, 8%, 9%, 10%, 11%, 12%, 13%, 14%, 15%, 16%, 17%<18%, 19%, 20% or more (w/w)).

In other examples, the amount of encapsulated inducing (or repressing) agent and encapsulated cells administered to the subject is determined based on a desired amount of release of the polypeptide encoded by the gene operably linked to the inducible (or repressible) promoter in the encapsulated cells for example, over a specified period of time (for example about 2 hours to 7 days or more, such as about 4 hours, 8 hours, 12 hours, 24 hours, 36 hours, 2 days, 3 days, 4 days, 5 days, 6 days, 7 days, or more). This can be adjusted, for example by increasing or decreasing the number of capsules including the cells, the number of cells per capsule, or the number of capsules including the inducing (or repressing) agent administered to the subject. The amount of released polypeptide from capsules including a known number of cells per capsule in the presence of a given concentration of inducing (or repressing) agent or a known concentration of inducing (or repressing) agent can be determined utilizing methods known to one of ordinary skill in the art, including for example, the methods described in Examples 2 and 3, below.

In other examples, the amount of encapsulated inducing (or repressing) agent and encapsulated cells administered to the subject is determined based on a desired amount of release of the inducing (or repressing agent) for example, over a specified period of time (for example about 2 hours to 7 days or more, such as about 4 hours, 8 hours, 12 hours, 24 hours, 36 hours, 2 days, 3 days, 4 days, 5 days, 6 days, 7 days, or more). This can be adjusted, for example by increasing or decreasing the number of capsules including the inducing (or repressing) agent or the loading of the capsules with the inducing (or repressing agent) administered to the subject. The amount of release of an inducing (or repressing) agent can be determined utilizing methods known to one of ordinary skill in the art, for example, the methods described in Example 3, below.

An effective amount of the delivery system can be the amount necessary to treat or inhibit a disease or disorder in a subject. The delivery system can be administered in a single dose, or in several doses, for example weekly, bi-monthly, or monthly during a course of treatment. One of ordinary skill in the art can determine the effective amount of the delivery system based for example, on the subject being treated, the severity and type of the affliction, and the manner of administration of the therapeutic(s).

A pharmaceutical composition that includes the delivery system (for example, including a pharmaceutically acceptable carrier) can be formulated in unit dosage form, suitable for individual administration of precise dosages. The amount of delivery system administered to the subject will be dependent on the particular inducing (or repressing) agent(s) and cells including regulatable gene(s) of interest, the subject being treated, the severity of the affliction, and the manner of administration, and is best left to the judgment of the prescribing clinician.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, implantable formulations usually comprise fluids that are pharmaceutically and physiologically acceptable fluid vehicles such as water, physiological saline, other balanced salt solutions, aqueous dextrose, glycerol or the like. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The disclosed delivery systems (such as encapsulated inducing or repressing agent, encapsulated cells, and/or co-encapsulated inducing (or repressing) agent and cells) can be administered to any suitable site in a subject. In some examples, the delivery system is implanted subcutaneously or intraperitoneally. In other examples, the delivery system is implanted at a site affected by a specific disease or disorder. In some examples, the delivery system is implanted in the central nervous system (for example, the striatum, cerebral cortex, subthalamic nuclei, or cerebrospinal fluid, such as the subarachnoid space or the lateral ventricles), the eye (for example, in the aqueous and/or vitreous humor), the kidney subcapsular space, or the synovial cavity of a joint (for example a joint affected by osteoarthritis). In other examples, the delivery system is administered or implanted at a wound site (for example, a skin wound or ulcer site or a bone fracture site). The delivery system can also be extracorporeal, such as included in an extracorporeal circuit (for example, included in a flow-through device).

### V. Kits

Disclosed herein are kits, which can be used for carrying out various embodiments of the disclosed methods. In some examples, the kits include an encapsulated inducing agent, an encapsulated repressing agent, or both. The kits also include an encapsulated plurality of cells which include a gene operably linked to an inducible promoter or a repressible promoter. In some examples, the encapsulated inducing agent and the encapsulated plurality of cells are included in separate containers. In other examples, the encapsulated inducing agent and the encapsulated plurality of cells are included in the same container. Similarly, in some examples, the encapsulated repressing agent and the encapsulated plurality of cells are included in separate containers. In other examples, the encapsulated repressing agent and the encapsulated plurality of cells are included in the same container. In some embodiments, the encapsulated inducing agent and the plurality of cells are co-encapsulated. In other embodiments, the encapsulated repressing agent and the plurality of cells are co-encapsulated.

In some examples, the kits also include at least one pharmaceutically acceptable buffer, for example in a separate container or in the same container as the encapsulated inducing (or repressing) agent and/or the encapsulated plurality of cells. The kits can also include instructions for utilizing the delivery system.

The present disclosure is illustrated by the following non-limiting Examples.

### EXAMPLES

### Example 1

### Continuous Delivery of IGF-1 from Encapsulated Cells

### Methods

Primary normal human fibroblasts (NHFs) were harvested from the dermis of neonatal foreskins (Women and Infants' Hospital, Providence, R.I.) with appropriate institutional review board approval and cultured as previously described (Campaner et al., J. Invest. Dermatol. 126:1168-1176, 2006). Except where stated otherwise, NHFs were cultured at 37° C. and 10% CO₂ in Dulbecco's modified Eagle medium (DMEM) supplemented with 10% fetal bovine serum (FBS, Invitrogen, Carlsbad, Calif.) and 1% penicillin/streptomycin (Invitrogen, Carlsbad, Calif.).

Normal human fibroblasts were genetically modified using a retroviral vector (MFG-IGF1) containing the human gene encoding IGF-1 as established by Eming et al. (J. Invest. Dermatol. 107:113-120, 1996) and the retroviral gene transfer technique established by Morgan et al. (Science 237:1476-1479, 1987). Briefly, the vector was transfected into a packaging cell line, and the resulting stable virus-producing cells were grown in a 10-cm Petri dish. Upon reaching confluence, culture medium was aspirated and fresh medium was added to the dish (10 ml/dish). After 24 hours, culture medium was removed, filtered using a 0.45-μm filter, and stored at −80° C. For transduction, 1.15×10⁵ NHFs (passage 4) were seeded in a 35-mm petri dish and cultured overnight. A thawed stock of retrovirus was incubated with 80 μg/ml of chondroitin sulfate C from shark cartilage (Sigma-Aldrich, St. Louis, Mo.) at 37° C. for 10 minutes, followed by incubation with 80 μg/ml of polybrene (1,5-dimethyl-1,5-diazaundecamethylene polymethobromide, hexadimethrine bromide, Sigma Aldrich) for an additional 10 minutes at 37° C. The mixture was centrifuged at 10,000 rpm for 5 minutes at room temperature, and the visible pelleted virus was isolated and resuspended to one-tenth of its original volume in cell culture medium. In the dish containing the NHFs, the culture medium was completely removed. The virus-containing culture medium was added and cells were incubated at 37° C. for 24 hours. Normal human fibroblasts were then washed and incubated with fresh culture medium for an additional 24 hours. Cells were trypsinized and plated in 10-cm tissue culture dishes. To assess the success of transfection, modified and unmodified cells were then grown to confluence in culture medium containing 1% FBS. For 48 hours post-confluence, conditioned medium was collected and assayed for concentration of IGF-1 using an enzyme-linked immunosorbent assay (ELISA, R&D Systems, Minneapolis, Minn.) and SPECTRAMAX® Absorbance plate reader (Molecular Devices, Sunnydale, Calif.).

Transfection was confirmed by the overexpression of IGF-1 from modified cells compared with unmodified controls. To determine if IGF-1 production was stable with cell passage, conditioned medium from cells from passages 6-10 was collected and assessed for concentration of IGF-1 using ELISA. Upon confirmation of a stable transfection, cells were frozen in liquid nitrogen at passages 6-7 to create a working cell bank.

After thawing, cells were expanded in tissue culture flasks before use. For all studies, passage 9-11 polyclonal cells were used. After expansion, cells were trypsinized and counted using a hemocytometer. Cells were then suspended in sterile filtered 1.8% alginate solution containing 0.9% sodium chloride at a target density of 1×10⁶ cells per ml of alginate. Using a commercially available encapsulator (Inotech, Dottikon, Switzerland), the cell suspension was extruded through a 100-mm nozzle at a vibration of approximately 5000 Hz and a flow rate of 1.8 ml per minute. Individual droplets were collected in a stirring bath of 0.15 M calcium chloride (Sigma-Aldrich) solution with 5 mM HEPES (pH 7.4); calcium ions cross-linked the alginate co-polymer chains, leading to the formation of Ca²⁺-alginate microcapsules. Capsules were allowed to gel and harden for 10 minutes, and then filtered from the bath using a 40-1 μm mesh strainer (BD Biosciences, San Jose, Calif.). Capsules were then washed three times with sterile DMEM and resuspended in culture medium.

Encapsulated cell viability was determined immediately after capsule fabrication, at the end of the 10-day release study, and at every time point in the proliferation studies. To calculate the cell viability, capsules were incubated in 3-(4,5-dimethylthiazol-2-yl)-2,5-diphenyltetrazolium bromide (MTT, Sigma-Aldrich) at 10 mg/ml in phosphate buffer solution (PBS, Invitrogen) for 4 hours. Alginate capsules were then solubilized via incubation in 55 mM sodium citrate solution containing 0.45% NaCl and 10 mM HEPES (pH 7.4) for 5 minutes. Cells were collected via centrifugation at 400 g for 5 minutes, resuspended in DMEM, and then counted using a hemocytometer. Live cells, which reduced the MTT dye to a dark purple formazan precipitate within their mitochondria, were stained purple, while necrotic cells remained unstained. The number of live and dead cells was counted, with viability represented as the ratio of live cells to the total number of cells encapsulated. The mean number of live cells per capsule was determined by dividing the number of live cells in an aliquot by the number of capsules in the aliquot.

For unencapsulated cell release studies, NHFs were grown to confluence in 6-well tissue culture plates containing DMEM with 10% FBS. At hours 12, 24, 36, and 48, the conditioned culture medium in each well was completely removed and fresh culture medium was added to the well. Conditioned medium samples were stored at −80° C. for later analysis. At the end of the study, cells were detached from the wells using 0.25% trypsin with EDTA (Invitrogen) and counted using a hemocytometer. For encapsulated studies, alginate capsules containing NHFs were incubated in 6-well tissue culture plates containing DMEM with 10% FBS. Each well was plated with approximately 50,000-100,000 encapsulated cells based on post-encapsulation cell count. At days 2, 4, 6, 8, and 10, each sample was transferred to a sterile 15-ml conical tube and centrifuged at 400 g for 5 minutes to isolate capsules from supernatant. The supernatant was then completely removed using care to not remove any capsules and fresh culture medium was added to the conical tube. Supernatant samples were stored at −80° C. for later analysis of IGF-1 content. At the end of the study, cell count and viability was assessed by incubating capsules with MTT dye before solubilization and counting with a hemocytometer.

The quantity of human IGF-1 from each supernatant sample was determined using ELISA. All samples were run in triplicate, and amount released was normalized to one million viable cells per sample as counted at the end of each release kinetics study. Fresh culture medium containing FBS was also measured for IGF-1 content to account for any cross-reactivity of bovine-derived IGF-1 with the ELISA. Profiles were plotted as cumulative mass release versus time, with all data points shown as mean±standard deviation. Release rates were determined by obtaining the slope from first-order linear regression forced through the axis on the profiles.

Encapsulated NHFs were incubated in 6-well tissue culture plates containing DMEM with 10% FBS. Each well was plated with approximately 25,000-50,000 cells based on the post-encapsulation cell count. Cell proliferation and viability within the capsules was assessed over the course of 7 days with culture medium changes occurring every 3 days. On days 0, 1, 3, 5, and 7, six wells of capsules were incubated with MTT dye before solubilization and cells were counted with a hemocytometer to determine the total number of cells in each well. The percent cell viability for the cells in each well was determined as the ratio of live cells to the total number of cells encapsulated. Cell viability is reported as mean±standard deviation; significance (p=0.01) was determined using a t-test assuming unequal variances.

A release study was completed using assay medium consisting of DMEM/F-12 (1:1; Invitrogen) supplemented with 1% penicillin/streptomycin, 0.2% bovine serum albumin (Sigma-Aldrich), and 10 μg/ml transferrin (Invitrogen). Alginate capsules containing NHFs were incubated in 6-well tissue culture plates containing bioactivity assay medium. At days 2, 4, 6, 8, and 10, all supernatant was completely removed in each well and replenished with fresh assay medium. Supernatant samples were stored at −80° C. for later analysis of IGF-1 content. All assay medium used on cells was analyzed via ELISA for IGF-1 concentration.

### Results

NHFs immobilized within Ca²⁺-alginate microcapsules were assessed by phase contrast microscopy. Cells were distributed at low density throughout approximately spherical capsules in a contracted form within the Ca²⁺-alginate matrix. Cells remained in this contracted state for the duration of culture. The cell packing density for the capsules was approximately 30-40 cells per capsule. Using Coulter particle sizing techniques, volumetric diameter was measured to be 457±172 μm. Capsule volume was calculated to be approximately 0.05 mm³ assuming spherical capsule geometry. Capsule size and loading cell density could be increased or decreased by adjusting the frequency of the vibration and adjusting the number of cells suspended in alginate prior to encapsulation. Assuming a nominal cell diameter of 20 μm, the total volume of a capsule occupied by the cells was approximately 0.2%-0.3%.

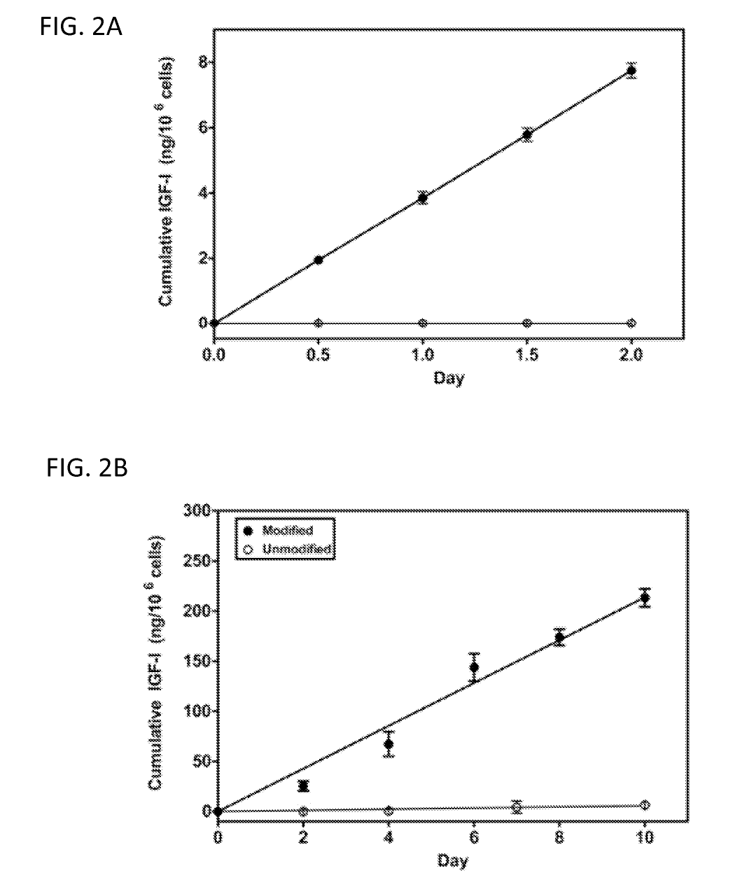

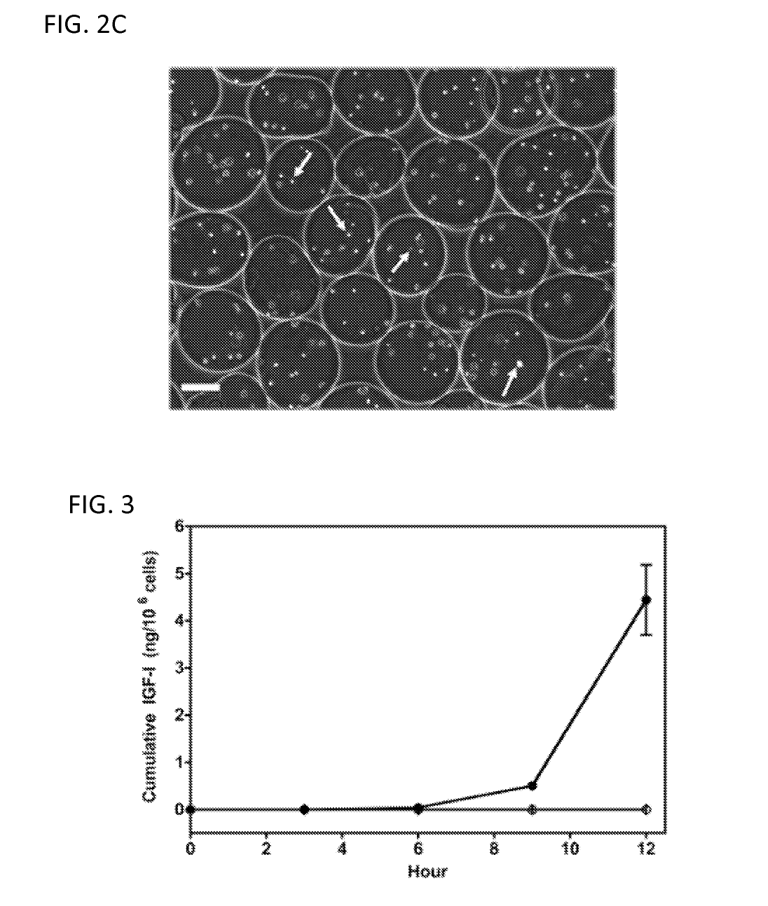

No IGF-1 was detected by ELISA in fresh culture medium containing 10% FBS. The cumulative release profiles of IGF-1 from the culture medium in which encapsulated and unencapsulated NHFs were cultured are shown in FIGS. 2A and B. Release is presented as cumulative mass released in culture medium as a function of time in culture, and this quantity is normalized to the number of cells counted in the culture at the end of the study. In unencapsulated culture, modified NHFs released 3.9±0.0 ng IGF-1 per 10⁶ cells per 24 hours, while unmodified NHFs released no IGF-1 as detected by ELISA. When encapsulated, modified and unmodified NHFs secreted 21.4±1.0 and 0.6±0.1 ng IGF-1 per 10⁶ cells per day, respectively. Encapsulated NHFs are shown in FIG. 2C. No significant changes in the number of both live and dead cells within capsules (no proliferation) were observed when measured daily over 7 days in vitro. In addition, initial and final counts remained the same for the 10 day release study. The released IGF-1 was bioactive as determined using an assay of growth of MCF-7 cells with conditioned medium from modified encapsulated NHFs.

### Example 2

### Regulated Delivery of IGF-1 from Encapsulated Cells

### Methods

Chinese hamster ovary cells (CHO-K1 Tet-On, Clontech Laboratories, Mountain View, Calif.), stably transfected to express the transactivator protein (rtTA) for a tetracycline inducible gene expression system, were cultured at 37° C. and 5% CO₂ in DMEM supplemented with 10% tetracycline-free FBS (Clontech), 1% penicillin/streptomycin (Invitrogen, Carlsbad, Calif.), and 200 μg/mL G418 (Sigma-Aldrich). All cell counting was done using Trypan Blue exclusion and the COUNTESS® Automated Cell Counter (Invitrogen).

To establish a stable cell line capable of tetracycline induced expression of IGF-1, a cDNA fragment containing the human IGF-1 gene was first isolated from a vector synthesized by Blue Heron Biotechnology (Bothell, Wash.) and inserted into the mammalian expression vector pTRE-Tight (Clontech) at the BamHI and HindIII sites. pTRE-Tight is a tetracycline-regulated expression vector that reduces background expression of the gene of interest. The pTRE-Tight-IGF1 construct and a linear hygromycin B selection marker (Clontech) were transfected into CHO-K1 Tet-On cells using FUGENE® 6 (Roche Applied Science, Indianapolis, Ind.) to generate a double stably transfected cell line, hereafter referred to as CHO-K1 Tet-IGF1. Transient tetracycline-inducible IGF-1 expression was assayed using 1 μg/mL of doxycycline hydrochloride (DOX, Clontech) one day post-transfection. Double stable clonal populations were then selected using 400 μg/mL of hygromycin B (Clontech).

The quantity of human IGF-1 from each supernatant sample was determined using ELISA (R&D Systems, Minneapolis, Minn.) and SPECTRAMAX® Absorbance plate reader (Molecular Devices, Sunnydale, Calif.). All samples were run in triplicate and amount released was normalized to one million viable cells per sample as counted at the end of each study.

Modified and unmodified cells were plated in 6-well tissue culture plates at a density of 3×10⁵ cells per well (n=6 per cell type). One day post-plating, cells were exposed to 1 μg/mL of DOX. At hours 3, 6, 9, and 12, conditioned medium was completely removed and stored at −20° C. for later measurement. Removed medium was replaced by fresh culture medium. At the end of the study, cells were trypsinized and counted.

Cells were encapsulated as described in Example 1. Microcapsule size was determined using a Coulter LS230 particle size analyzer (Beckman Coulter, Fullerton, Calif.). Measured diameter was used to determine the volume of a capsule assuming uniform spherical geometry. The total number of capsules in a sample was determined visually using a 0.1 mL aliquot of the original sample and light microscopy.

Encapsulated cell count and viability (live cells per total number of cells encapsulated) were determined by solubilizing capsules in 55 mM sodium citrate (Sigma Aldrich) with 0.45% NaCl and 10 mM HEPES for one minute. Cells were collected via centrifugation, resuspended in culture medium, and counted. There was no significant difference between percent viabilities obtained pre- or post-solubilization of the capsules, and as a result, Trypan Blue exclusion was utilized to determine percent cell viability post-solubilization.

Modified encapsulated and unencapsulated cells were plated into 12-well tissue culture plates. One day post-plating, cells were exposed to DOX (n=3 per cell type per DOX dose). After 24 hours, conditioned medium was completely removed and stored at −20° C. for later measurement using ELISA. At the end of the study, cells were trypsinized and counted.

Encapsulated modified and unmodified cells were plated in 12-well tissue culture plates at an equal density per well and cultured at either 32° C. or 37° C. Cells were assessed for cell number and viability on days 0, 2, 4, and 6 for 32° C. cultures and days 0, 1, 2, and 3 for 37° C. cultures (n=3 per time point).

Encapsulated modified and unmodified cells were plated in 12-well tissue culture plates at an equal density per well and cultured at 32° C. (n=11). On days 0-2, cultures were exposed DOX (1 μg/mL). On days 3-10, DOX was absent from cultures. At every time point, conditioned medium was completely removed from each well and replaced with fresh culture medium. Samples were stored at −20° C. for later IGF-1 quantification using ELISA. To ensure all DOX was removed from the culture on day 3, each culture was washed three times with DOX-free medium before being plated in new 12-well plates. On day 10, capsules were solubilized and cells were counted.

### Results

After 6 hours of continuous exposure to DOX, modified CHO-K1 Tet-IGF1 cells began to release IGF-1 at levels that were detectable by ELISA, and after 12 hours of continuous DOX exposure, a total of 5 ng of IGF-1 per 10⁶ cells was released. Release was not constant, with release rates increasing over the time of exposure (FIG. 3). Unmodified controls did not release detectable levels of IGF-1 for the duration of the study.

Phase contrast microscopy indicated that the cells remained in a contracted form for the duration of culture and the majority of the capsules were spherical in nature. Intracapsular cell density was approximately 74 cells/capsule and the mean capsule diameter was measured to be 585.9±153.2 μm. Assuming a spherical geometry, mean capsule volume was calculated to be 0.1 mm³.

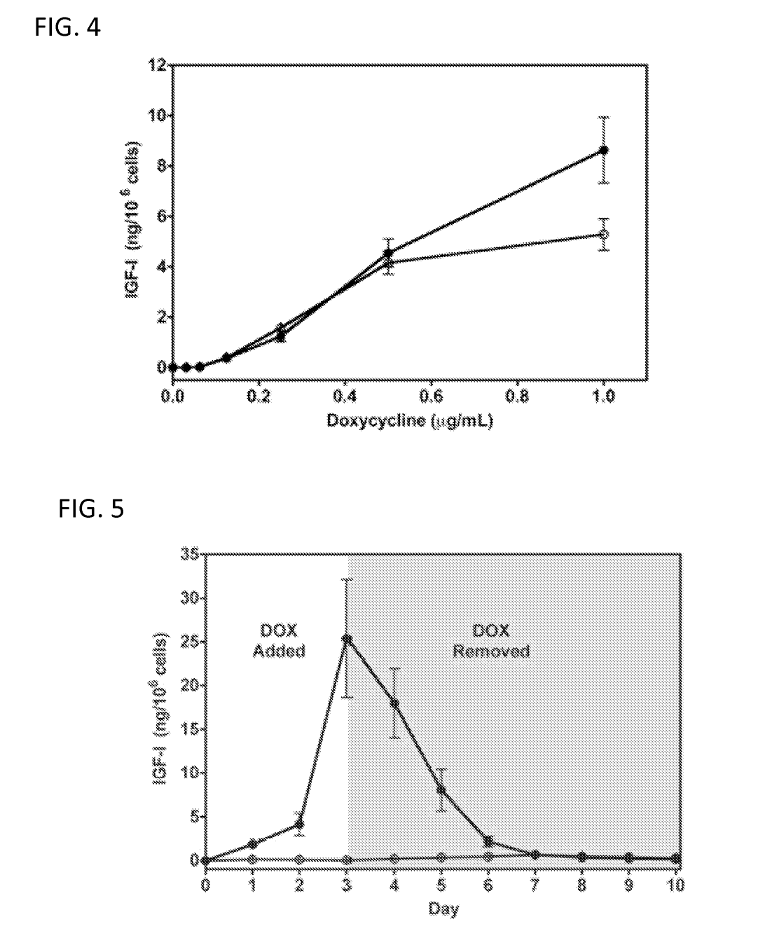

Encapsulated and unencapsulated Cho-K1 Tet-IGF1 cells were capable of inducible IGF-1 release that was dependent on DOX concentration (FIG. 4). Both encapsulated and unencapsulated cells released IGF-1 at detectable levels with exposure to DOX concentrations of 0.125 μg/mL or higher, while DOX concentrations lower than this value yielded no detectable IGF-1 release. Dose-dependent IGF-1 release was consistent for both encapsulated and unencapsulated cultures for all DOX concentrations studied, with the exception of 1 μg/mL. At this concentration of DOX, encapsulated cells released IGF-1 at a level that was 1.6-fold higher than that of unencapsulated cells. Additionally, for DOX concentrations of 0.125 μg/mL or higher, IGF-1 release from encapsulated cells was constant or constitutive with respect to DOX concentration with a release rate of 6.7±0.3 ng of IGF-1 per 10⁶ encapsulated cells per μg of DOX. There were no significant differences in proliferation or viability between the modified and unmodified encapsulated cells. The released IGF-1 was bioactive as determined using an assay of growth of MCF-7 cells with conditioned medium from modified encapsulated NHFs.

The ability to activate and deactivate IGF-1 genetic expression and subsequent release from encapsulated cells using DOX was demonstrated with a 10 day release study conducted at 32° C. (FIG. 5). DOX was present from day 0 through day 3 of the study. Correspondingly, IGF-1 was released from encapsulated modified cells starting at day 1 and continuing up through day 3. On day 3 onwards, DOX was removed from the cultures, causing the IGF-1 release levels to decrease exponentially over time, with a decay half-life of 0.83 days. IGF-1 release reached levels comparable to those of unmodified cells by day 7. The maximal daily release of IGF-1 for modified cells occurred on day 3 with an average release of 25.4±6.8 ng of IGF-1 per 10⁶ encapsulated cells. For days 7 through 10, there was no statistical difference observed in IGF-1 release for modified and unmodified cells (p=0.01). The released IGF-1 was bioactive as determined using an assay of growth of MCF-7 cells with conditioned medium from modified encapsulated NHFs.

### Example 3

### Regulated Delivery of IGF-1 from Co-Encapsulated Cells and Polymeric Microspheres

### Methods

Cho-K1 Tet-IGF1 cells were prepared and cultured as described in Example 2. Poly(L,D-lactic-co-glycolic-acid) (PLGA 50:50; Resomer RG502, B oehringer Ingleheim, Germany) microspheres were fabricated using an encapsulation water/oil/oil (W/O/O) double emulsion solvent removal method. All solvents were purchased from Fisher Scientific (Hampton, N.H.) and were of the highest commercial grade available. Silicon oil (100 CST) was obtained from Dow Corning (Midland, Mich.). In brief, 500 mg of PLGA was dissolved in 15 mL of dichloromethane (DCM). Five mg of doxycycline hydrochloride (DOX, Clontech) was dissolved in 300 μL of distilled water to achieve a final theoretical formulation of 1% dry weight of the drug to the dry weight of the polymer (w/w). A primary W/O emulsion was generated by first adding the aqueous DOX solution to the polymer solution and then probe sonicating the resultant mixture for one minute using an Ultrasonic Homogenizer CV26 (Cole-Parmer, Vernon Hills, Ill.) at 40% amplitude. The resulting emulsion was then added to 80 mL of a 20% v/v DCM/silicon oil solution and probe sonicated for 2 minutes to generate the secondary W/O/O emulsion. This double emulsion was then poured into a 1 L bath of petroleum ether and stirred at 2000 RPM for 5 minutes to allow for solvent removal and phase inversion of the polymer to form PLGA microspheres containing DOX. Spheres were collected using a positive pressure filtration column with a 0.2 μm PTFE filter (Millipore, Billerica, Mass.) and further washed with petroleum ether to remove the silicon oil. The final product was flash frozen, lyophilized for 2 days, and stored at −20° C. Unloaded PLGA microspheres were also fabricated using the above method and served as a negative control.

To determine the encapsulation efficiency of DOX loaded PLGA microspheres, 10 mg of microspheres (n=3) were dissolved in 2 mL of 0.1 N NaOH for 24 hours in the dark. The resulting solution was filtered using a 0.2 μm PTFE syringe filter (National Scientific, Rockwood, Tenn.) and DOX concentration was determined with UV spectrophotometry at an absorbance of 255 nm using a SPECTRAMAX® Absorbance plate reader and SOFTMAX® Pro software (Molecular Devices, Sunnydale, Calif.). All samples were run in triplicate and data are reported as mean±standard deviation. Encapsulation efficiency was defined as the ratio of actual DOX loading to the theoretical maximum loading.

Scanning electron microscopy (SEM) was utilized to assess particle sizes and evaluate the surface morphology. Samples were mounted to a carbon-backed adhesive, sputter coated with gold-palladium for 6 minutes at 20 mA with an Emitech K550 sputter coater (West Sussex, United Kingdom), and visualized using a Hitachi S-2700 scanning electron microscope (Hitachi, Peoria, Ill.) with an accelerating voltage of 8 kV. Microsphere size was analyzed using a Beckman-Coulter LS230 Laser Diffraction Particle Size Analyzer (Beckman-Coulter). Prior to Coulter analysis, 5-7 mg of spheres were suspended in an aqueous solution of 1% polyvinyl pyrrolidone (PVP; Sigma Aldrich) and 1% sodium lauryl sulfate (SLS; Sigma Aldrich) and sonicated to break up particle aggregation.

DOX release from microspheres was quantified over the course of 85 days in vitro. About 10 mg of microspheres (n=6) were suspended in 1.25 mL of phosphate buffered saline (PBS, pH 7.4, Invitrogen) and incubated at 37° C. and 5% CO₂. At every time point (1, 2, 4, and 8 hours; 1, 2, 3, 5, 7, 14, 21, 28, 35, 42, 49, 56, 63, and 85 days), microspheres were removed from the incubator and centrifuged at 10,000 RPM for 5 minutes. One mL of supernatant was then removed and stored at −20° C. for later measurement. One mL of fresh PBS was then added to the microspheres. Spheres were re-suspended by vortexing briefly and returned to the incubator until the next time point. Prior to quantifying the DOX concentration in the collected samples using UV spectrophotometry, each solution was filtered using a 0.2 μm PTFE syringe filter to remove any debris. All samples were run in triplicate and data is shown as mean±standard deviation. Fractional release at each time point was normalized to the total mass released on the last day of the study.

Cells were encapsulated and encapsulated cell count and viability were determined as described in Examples 1 and 2. Microencapsulated and unencapsulated CHO-K1 Tet-IGF1 cells were co-cultured with DOX loaded PLGA microspheres to assess DOX-inducible IGF-1 synthesis and delivery. Approximately 5×10⁵ encapsulated or 5×10⁴ unencapsulated cells were plated per well in 12-well plates. After incubating cultures for 24 hours at 37° C., medium was completely removed from each well and 2 mL fresh media containing one of the following was added to each well (n=3): 1 μg/mL DOX (Clontech), 1.6 mg of DOX loaded PLGA microspheres, 1.6 mg of unloaded PLGA control microspheres, or no DOX. The mass of spheres used in these studies was calculated to release a total of 2 μg of DOX after 24 hours of incubation as determined by the results of the cumulative in vitro release. Cultures were incubated for another 24 hours, at which time the supernatant from each culture was removed and stored at −20° C. for later measurement. For unencapsulated cultures, cells were trypsinized and counted using Trypan Blue exclusion. For encapsulated cultures, cells were collected via alginate solubilization prior to counting. IGF-1 was quantified as described in Example 2.

Cells and PLGA microspheres were co-encapsulated within Ca²⁺-alginate macrocapsules. Twenty mg of PLGA microspheres were first added to 0.5 mL of 1.25% sorbitol (Sigma Aldrich) buffer containing 0.025% Tween® 20 (Sigma Aldrich), 0.25% hydroxypropyl methylcellulose (HPMC; Dow Corning), and 0.9% sodium chloride and briefly sonicated to reduce particle aggregation. This suspension was then added to 1.25% sodium alginate with 20 mM HEPES and 150 mM sodium chloride to obtain a final microsphere density of 2 mg/mL. The alginate suspension was briefly vortexed to ensure adequate dispersion, and then cells were added at a density of 2×10⁶ cells per mL of alginate and dispersed within the alginate suspension via pipetting. The resulting cell-microsphere suspension was loaded into a 10-mL sterile syringe (BD Biosciences, Bedford, Mass.) and passed through a 20-gauge sterile needle (BD Biosciences) using a syringe pump (Harvard Apparatus, Holliston, Mass.) at a speed of 1.5 mL/min. Resulting droplets were collecting in a stirring bath of 102 mM calcium chloride with 10 mM HEPES. Capsules were allowed to harden for 10 minutes, filtered and washed with DMEM, and then resuspended in culture medium. Using this encapsulation method, macrocapsules containing only PLGA microspheres were also prepared. After fabrication, these macrocapsules were washed and stored in PBS for further study. Macrocapsules containing unloaded PLGA microspheres were also fabricated.

Macrocapsule size was determined using light microscopy. Diameters were measured from microscopy images using Image J software (NIH, Bethesda, Md.) and used to determine the volume of a capsule assuming uniform spherical geometry. The number of capsules per sample was hand counted. To determine the encapsulated cell count and viability (live cells per total number of cells encapsulated), macrocapsules were solubilized by introduction to a 55 mM EDTA solution with 10 mM HEPES for five minutes at 37° C. with gentle agitation. Cells were then collected via centrifugation, resuspended in culture medium, and counted using Trypan Blue exclusion.

DOX release from Ca²⁺-alginate macrocapsules containing only PLGA microspheres was quantified over the course of 7 days in culture using 12-well tissue culture plates. Approximately 150 macrocapsules in 2 mL of PBS were added per well (n=6) and the samples were incubated at 37° C. and 5% CO₂. At 24-hr intervals, the samples were removed from the incubator and the supernatant in each well was completely removed and stored at −20° C. for later measurement. Two mL of fresh PBS was added to the well and the samples were returned to the incubator until the next time point. Prior to quantifying the DOX concentration in the collected samples using UV spectrophotometry, each solution was filtered using a 0.2 μm PTFE syringe filter to remove any debris. All samples were run in triplicate and data are shown as mean±standard deviation and normalized to the number of capsules per sample as counted at the end of the study. Macrocapsules containing unloaded PLGA microspheres served as a negative control.

DOX-induced IGF-1 synthesis and delivery from alginate macrocapsules containing both cells and PLGA microspheres was quantified over the course of 7 days in culture using 12-well tissue culture plates. Approximately 50 macrocapsules in 2 mL of culture medium were added per well (n=6) and incubated at 37° C. and 5% CO₂. At 24-hr intervals, the samples were removed from the incubator and the supernatant from each well was completely removed and stored at −20° C. for later assay. Two mL of fresh culture media was added to each well and the samples were returned to the incubator until the next time point. IGF-1 content in the supernatant samples was quantified using ELISA and was normalized to the number of cells and number of capsules per sample as counted at the end of the study. Macrocapsules containing cells and unloaded PLGA microcapsules served as a negative control.

### Results

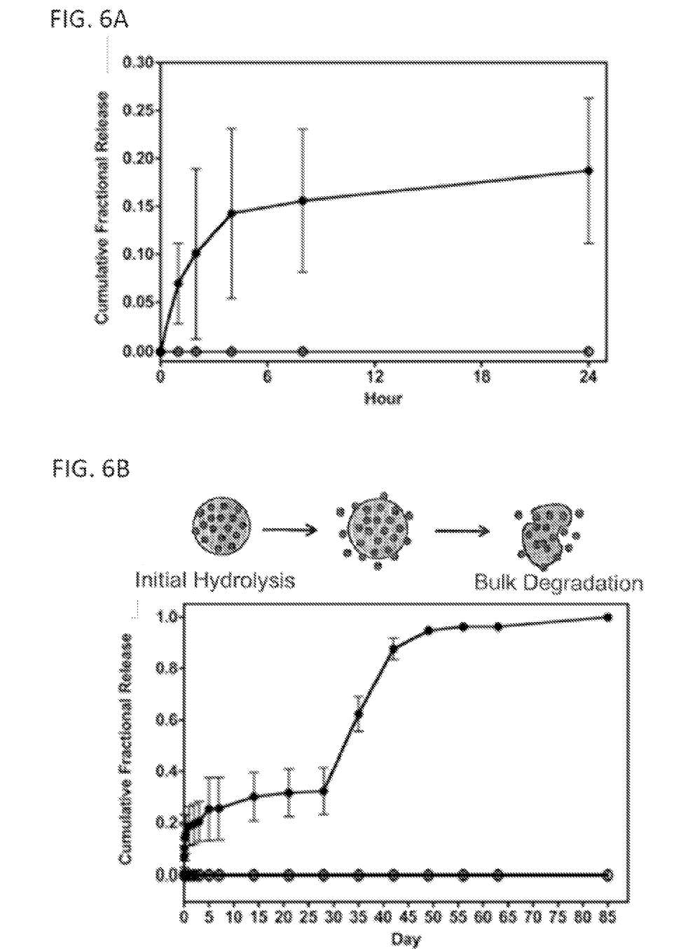

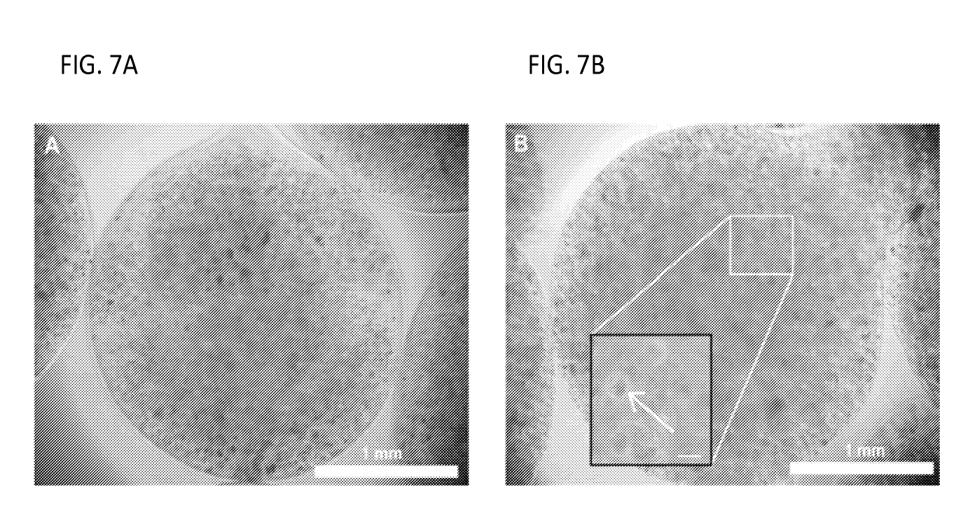

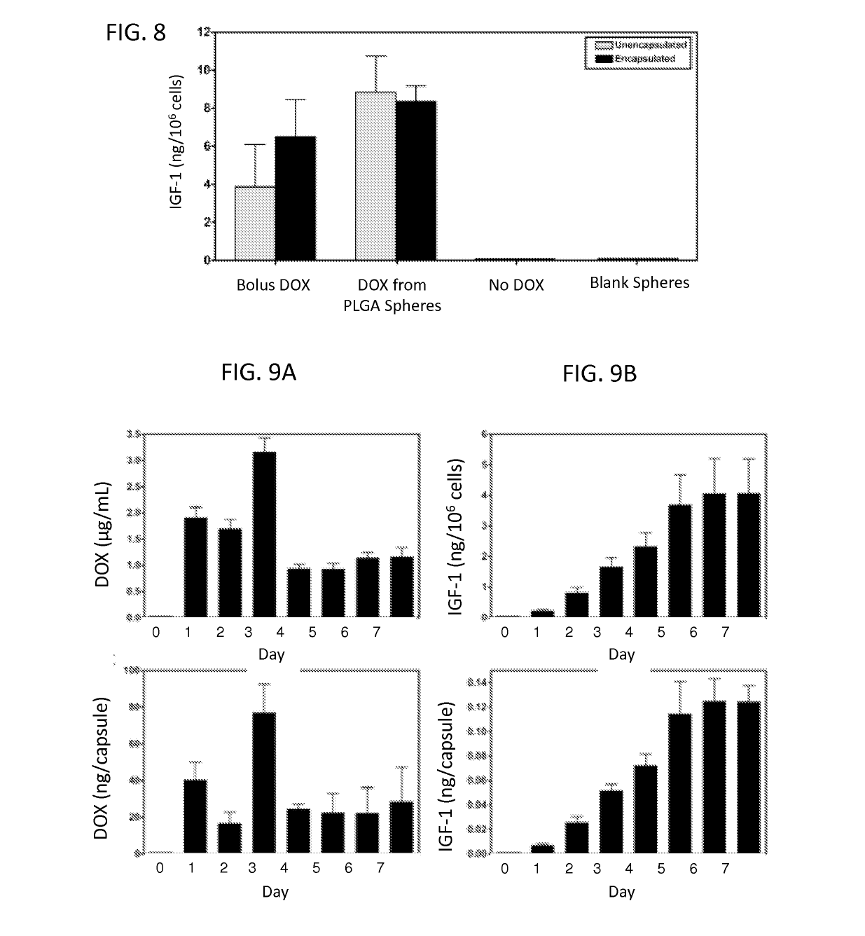

Using a W/O/O double emulsion solvent removal encapsulation method (Patel et al., J. Tissue Eng. Regen. Med. doi: 10.1002/term. 546, 2012; incorporated herein by reference), DOX loaded PLGA microspheres were produced with a 50:50 PLGA copolymer and a 1% w/w drug loading. The encapsulation efficiency of DOX was nearly 70%. Particle sizing indicated two populations of spheres in the micro- and nano-scale for both loaded and unloaded formulations (Table 1). This bi-modal distribution of particle sizes was further confirmed by SEM. For both unloaded and loaded formulations, a few larger, spherical microspheres were observed. A substantial number of aggregates consisting of smaller, spherical nano- and microspheres were also observed. DOX loading did not significantly change the surface morphology. All formulations had an intact outer surface, with small micropores observed on some of the larger microspheres. Fabricated PLGA microspheres had sustained release of DOX over the course of 85 days in vitro. Approximately 20% of the drug was released in the first 24 hours of study (FIG. 6A) and over the course of study, drug loaded microspheres exhibited biphasic release (FIG. 6B).

TABLE 1
Encapsulation efficiency and mean particle
size of PLGA microsphere formulations.

| Formulation | % Encapsulation Efficiency | Mean Particle Size |
|---|---|---|
| 1% DOX loaded | 69.6 ± 1.05 | 796 ± 623 nm; |
| | | 14.56 ± 11.79 μm |
| Unloaded | NA | 768 ± 635 nm; |
| | | 16.75 ± 12.99 μm |

CHO-K1 Tet-IGF1 cells were successfully encapsulated within Ca²⁺-alginate microcapsules and co-cultured with PLGA microspheres. Cells remained in a contracted form for the duration of culture and the majority of the capsules were spherical in nature with approximately 70 cells per capsule. Encapsulated and unencapsulated cells were co-cultured with DOX loaded PLGA microspheres to quantify DOX-induced IGF-1 synthesis and delivery after 24 hours in culture (FIG. 12). The total mass of spheres co-cultured with cells was chosen based upon the cumulative in vitro release after 24 hours to release a total of 2 μg of DOX after 24 hours. For comparison, three other conditions were also examined: bolus delivery of 2 μg of DOX at the beginning of the study, no DOX delivery, and co-culture with unloaded control PLGA microspheres. In the case of DOX delivery, via bolus or microsphere, IGF-1 synthesis and delivery was induced in both unencapsulated and encapsulated cultures. There was no significant difference between the amount of IGF-1 synthesized and delivered from encapsulated and unencapsulated cells for both DOX delivery formats. Cells cultured in media containing no DOX or co-cultured with unloaded control microspheres did not produce any detectable levels of IGF-1.

PLGA microspheres were successfully encapsulated within Ca²⁺-alginate macrocapsules (FIG. 11A). Microspheres were uniformly dispersed throughout the capsules with few aggregates. Microspheres could also be co-encapsulated with CHO-K1 Tet-IGF1 cells to create a combined capsule system consisting of both spheres and cells (FIG. 11B). In the combined system, both cells and spheres were evenly dispersed throughout the alginate hydrogel with approximately 1.7×10⁴ cells encapsulated per macrocapsule. Post-encapsulation cell viability was calculated to be over 88%. Fabricated macrocapsules had an average diameter of 2.5±0.16 mm, and assuming spherical geometry, a mean capsule volume of 7.9±1.61 mm³.

DOX had a sustained release from PLGA microspheres over the course of 7 days in culture, with a peak release at day 3 followed by a steady release rate from day 4 through 7 (FIG. 13A). Correspondingly, DOX-induced IGF-1 release from encapsulated cells was observed to occur starting on day 1 and increasing over time, reaching a steady rate of release after 5 days (FIG. 13B). Correspondingly, the average release rate for DOX from day 4 through 7 was 24.4±11.80 ng of DOX per capsule per day, while the average release rate for IGF-1 from days 5 through 7 was 0.1 ng±0.02 of IGF-1 per capsule per day.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A drug delivery system comprising:
an encapsulated inducing agent or repressing agent; and
an encapsulated plurality of cells, wherein the cells comprise a gene operably linked to a promoter inducible by the inducing agent or repressible by the repressing agent, and wherein the expression of the gene is modified by the inducing agent or the repressing agent.

2. The drug delivery system of claim 1, wherein the encapsulated inducing agent and the encapsulated plurality of cells are co-encapsulated.

3. The drug delivery system of claim 1, wherein the encapsulated inducing agent or repressing agent is encapsulated in one or more microspheres comprising a polymer.

4. The drug delivery system of claim 3, wherein the polymer comprises poly(lactic-co-glycolic acid) (PLGA).

5. The drug delivery system of claim 1, wherein the encapsulated plurality of cells is encapsulated in a microcapsule comprising a polymer.

6. The drug delivery system of claim 5, wherein the polymer comprises alginate, collagen, chitosan, gelatin, agarose, or a combination of two of more thereof.

7. The drug delivery system of claim 1, wherein the promoter inducible by the inducing agent comprises a tetracycline inducible promoter.

8. The drug delivery system of claim 7, wherein the inducing agent comprises tetracycline (TET) or doxycycline (DOX).

9. The drug delivery system of claim 1, wherein the promoter repressible by the repressing agent comprises a tetracycline repressible promoter.

10. The drug delivery system of claim 9, wherein the repressing agent comprises tetracycline (TET) or doxycycline (DOX).

11. The drug delivery system of claim 1, wherein the gene operably linked to the promoter encodes a growth factor.

12. The drug delivery system of claim 11, wherein the gene operably linked to the promoter encodes insulin-like growth factor-1 (IGF-1), insulin-like growth factor-2 (IGF-2), platelet derived growth factor (PDGF), fibroblast growth factor (FGF), keratinocyte growth factor (KGF), epidermal growth factor (EGF), transforming growth factor-α (TGF-α), transforming growth factor-β (TGF-β), vascular endothelial growth factor (VEGF), or granulocyte macrophage colony stimulating factor (GM-CSF).

13. The drug delivery system of 11, wherein the growth factor expression is induced by TET or DOX.

14. A method of treating or inhibiting a disease or disorder in a subject, comprising administering to the subject an effective amount of the drug delivery system of claim 1, thereby treating or inhibiting the disease or disorder.

15. The method of claim 14, wherein the subject is a mammal.

16. The method of claim 15, wherein the disease or disorder is selected from the group consisting of osteoarthritis, Parkinson's disease, Alzheimer's disease, Huntington's disease, amyotrophic lateral sclerosis, retinal degeneration, macular degeneration, diabetes, a tumor, and a wound.

17. The method of claim 16, wherein the disease or disorder is a wound, wherein the wound comprises a bone fracture or a skin ulcer.

18. The method of claim 17, further comprising administering to the subject an effective amount of an additional therapeutic agent.

19. A kit for treating a subject with a disease or disorder, comprising the drug delivery system of claim 1, in a container.

20. A drug delivery system comprising:
a plurality of poly(lactic-co-glycolic acid) (PLGA) microspheres encapsulating tetracycline or a tetracycline analog; and
a plurality of cells comprising an insulin-like growth factor 1 gene operably linked to a tetracycline-inducible promoter,
wherein the plurality of PLGA microspheres and the plurality of cells are co-encapsulated in a calcium-alginate macrocapsule.
